(19) United States
(12) Patent Application Publication
(10) Pub. No.: US 2014/0170588 A1
(43) Pub. Date: Jun. 19, 2014

(54) DENTAL LASER-EMITTING DEVICE AND METHODS

(71) Applicants: ALAN MILLER, INDIANAPOLIS, IN (US); BART WACLAWIK, CARMEL, IN (US); WILLIAM S. PARKER, ANN ARBOR, MI (US)

(72) Inventors: ALAN MILLER, INDIANAPOLIS, IN (US); BART WACLAWIK, CARMEL, IN (US); WILLIAM S. PARKER, ANN ARBOR, MI (US)

(73) Assignee: DENTSPLY INTERNATIONAL INC., YORK, PA (US)

(21) Appl. No.: 13/713,024

(22) Filed: Dec. 13, 2012

(51) Int. Cl. A61C 1/00; A61C 8/00; A61C 17/02; A61B 10/02; A61N 5/06; A61C 3/02; A61B 18/20; A61C 5/02

(52) U.S. Cl. CPC A61C 1/0046; A61C 1/0092; A61B 18/20; A61C 8/0089; A61C 5/02; A61B 10/02; A61N 5/0603; A61C 3/02; A61C 1/0023; A61C 17/0202
USPC 433/29; 433/215; 433/80

(57) ABSTRACT

Disclosed herein is a dental laser-emitting device capable of treating both soft tissue applications and hard tissue applications.

### PRIORITY INFORMATION

The present application claims priority to U.S. patent application Ser. No. 13/305,074, filed Nov. 28, 2011, which claims priority to U.S. Provisional Application No. 61/417,685 filed on Nov. 29, 2010.

### TECHNICAL FIELD

This invention relates to the field of medical lasers and, in particular, lasers used in the provision of dental treatment of hard tissue and soft tissue, including gingival tissue, skin, muscle, connective tissue, bone, tooth enamel, and tooth dentin.

### BACKGROUND AND SUMMARY

During dental procedures, it may be necessary to utilize various surgical techniques on hard tissue and soft tissue in treatment areas in and around the oral cavity. Such techniques may include the cutting and/or removal of either soft or hard tissue. In the past, various traditional surgical tools, such as scalpels, have been utilized to accomplish these techniques. In addition, medicines and antibiotics have been utilized for control of pain, as well as a preventive measure to avoid infection.

In the late 1950's, the high speed air rotor was developed for the removal of dental hard tissue, including enamel, dentin and dental caries. The high speed air rotor offered faster removal of hard tissue while also being more comfortable for the patient and easier to use for the dentist, compared to available electric belt drive dental drills. While offering advantages, the high speed air rotor was found to create excessive heat and high frequency vibration which was injurious to the vital tissues in the tooth; and a water spray or water misting system was developed in parallel with the high speed air rotor. The water spray or water mist was directed toward the operative site while the air rotor was spinning and a burr was in contact with tooth structure, thus safely cooling the tooth structure and dampening the injurious high frequency vibration.

Later, mid-infrared lasers became available for the removal of dental hard tissues by means of ablation. These lasers also used a water spray or water mist for cooling of the tooth structures and as a medium which absorbed the mid-infrared wavelength energy emitted by the lasers, thus enhanced the ablation of the dental hard tissues.

Laser-emitting devices are beginning to achieve increased popularity as tools to perform the above-described functions. Such laser-emitting devices may be used to cut and cauterize skin, including treatment areas on or around the lips and gums, and high power laser-emitting devices may be used to ablate bone, tooth dentin and tooth enamel. Laser-emitting devices may further be used in the debridement, denaturalization and sterilization of root canal surfaces. There are many benefits to using a laser-emitting device over traditional methods of performing these operations, including a significant reduction in the post-operative healing time, improved control over bleeding due to the simultaneous cauterization of the soft-tissue at the time of cutting, the opportunity to provide less-invasive treatments by making smaller and more precise cuts, the ability to treat with less anesthesia and possibly no anesthesia, the ability to gain access to and effectively treat otherwise inaccessible areas (e.g., sterilization and debridement of necrotic tissue, such as within periodontal pockets), and promotion of a potentially better surface for subsequent bonding procedures due to the lessened need to chemically etch tooth surfaces after drilling.

While there may be significant benefits associated with the use of a laser-emitting device to perform the above-mentioned treatments, there are also significant challenges. Dental lasers have taken considerable time to find adoption within the community of dental practitioners for a variety of reasons, including cost, the learning curve required to effectively use such devices, complicated setup parameters, difficulty in diagnosis of malfunctioning equipment, limited treatment applications for earlier designs, and institutionalized treatment methods that stayed relatively static for nearly a century, to name just a few. While cost tends to decline as a technology matures, other factors can be significantly mitigated through improvements in the design of the laser-emitting devices, including those described herein.

In one exemplary embodiment of the present invention; a laser-emitting device is described which comprises a housing, a power supply, two or more laser light sources, a controller configured to modulate one or more of the laser light sources; a memory operatively coupled to the controller to store device settings; a connection used to operatively couple a smart device to the controller, a handpiece for applying laser light to the area of treatment, an airless misting unit to apply a fine water mist to the area of treatment, and an articulated arm operatively coupling the laser light source to the handpiece.

### BRIEF DESCRIPTION OF THE DRAWINGS

The detailed description particularly refers to the accompanying figures, in which:

FIG. 1A is an isometric view of an exemplary embodiment of the dental laser-emitting device described herein;

FIG. 1B is a detail view of an exemplary embodiment of a secondary visual display described herein;

FIG. 2 is a front elevation view of an exemplary embodiment of the dental laser-emitting device described herein;

FIG. 3 is a side elevation view of an exemplary embodiment of the dental laser-emitting device described herein;

FIG. 4 is a top elevation view of an exemplary embodiment of the dental laser-emitting device described herein;

FIG. 5 is a rear elevation view of an exemplary embodiment of the dental laser-emitting device described herein;

FIG. 6 is an schematic depicting the components of an exemplary embodiment of the dental laser-emitting device described herein;

FIG. 7 is a detailed view of the laser light subsystem of an exemplary embodiment of the dental laser-emitting device described herein;

FIG. 8 is a diagram depicting the components of the airless misting unit described herein;

FIG. 9 is a line drawing depicting an exemplary embodiment of the user interface for the dental laser-emitting device described herein;

FIG. 10 is a flow chart depicting one exemplary embodiment of a method wherein the controller responds to user input;

FIG. 11 is a flow chart depicting one exemplary embodiment of a method wherein a diagnostic program is executed.

### DETAILED DESCRIPTION

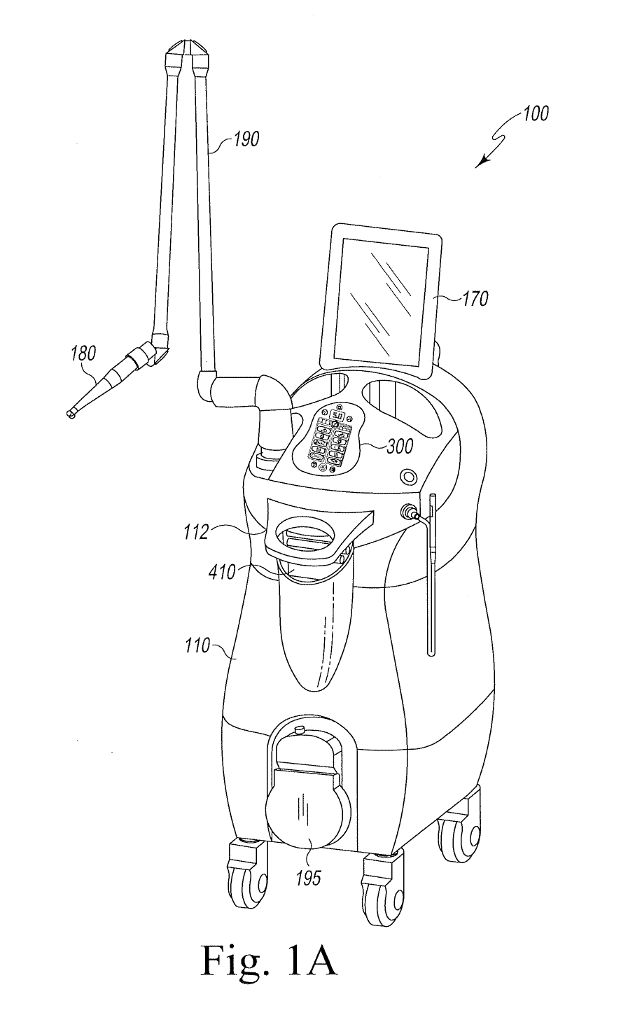

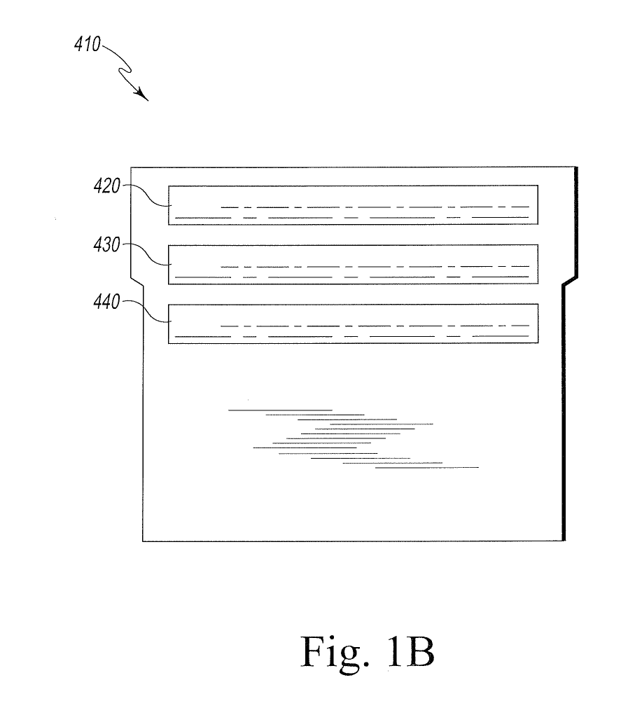

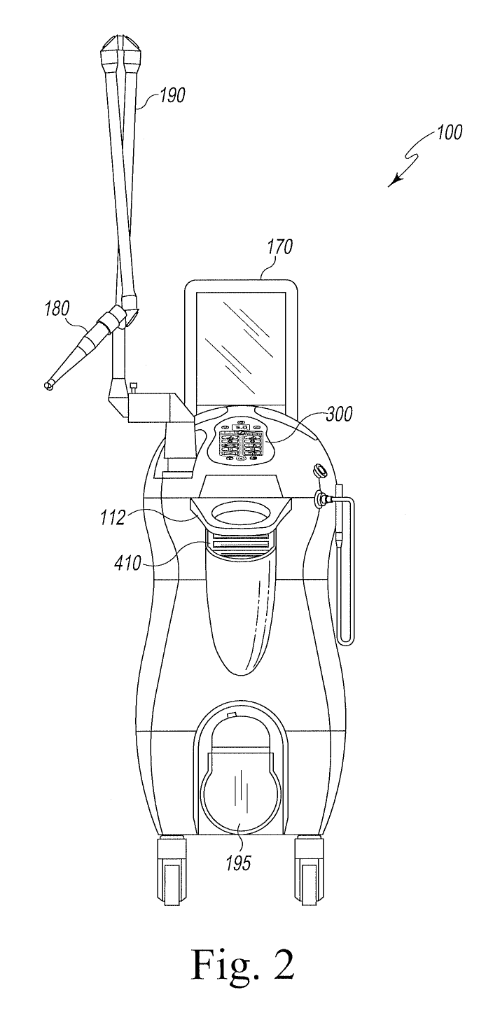

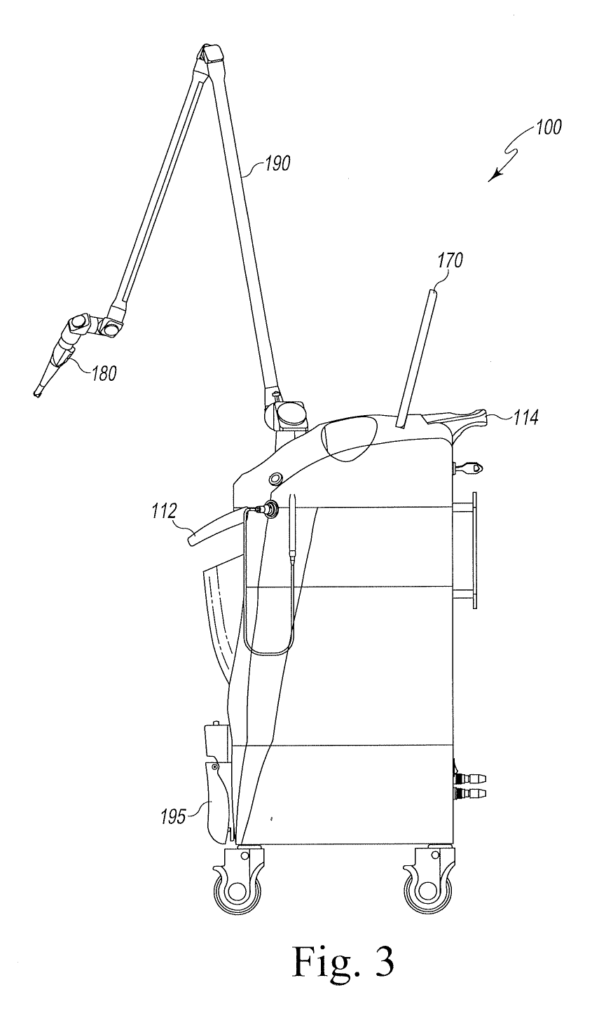

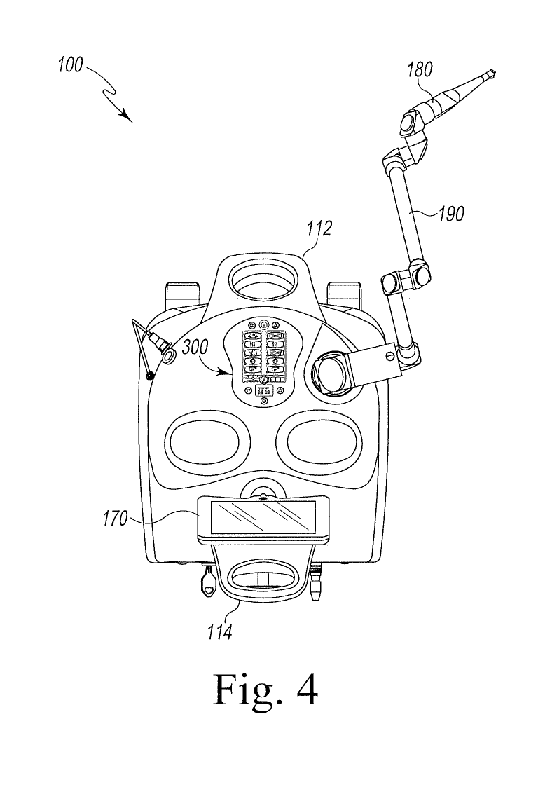

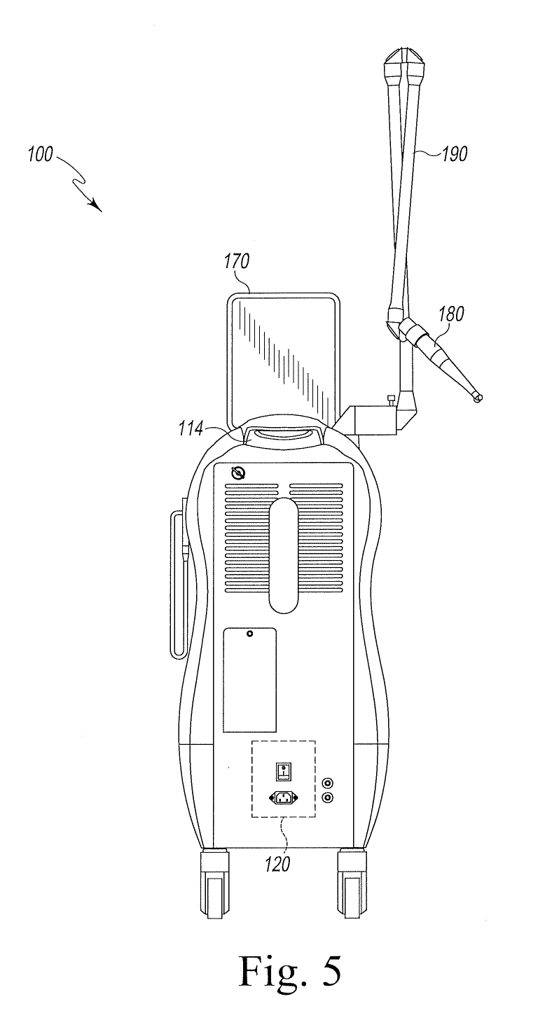

Referring now to FIGS. 1-5, laser-emitting device 100 includes a housing 110; a power supply 120; laser subsystem 130; a controller 140 configured to modulate one or more of the laser light sources; a memory 150 operatively coupled to the controller to store device settings; a connection 160 used to operatively couple a smart device 170 to the controller; a handpiece 180 for applying laser light to the area of treatment, an airless misting unit 200 to irrigate the area of treatment; an articulated arm 190 operatively coupling laser subsystem 130 to handpiece 180; and a user interface 300 located on the exterior of housing 110 or, alternatively, on smart device 170 operatively coupled to controller 140 through connection 160 and that allows an operator to modify the operational parameters of the laser subsystem 130 and/or airless misting unit 200. In the illustrative embodiment, housing 110 includes a front handle 112 and a rear handle 114.

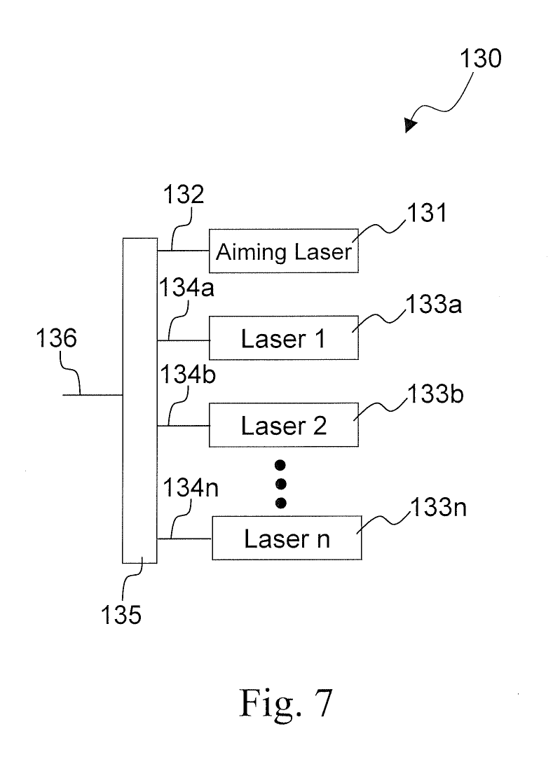

An exemplary embodiment of the laser subsystem 130 is depicted in FIG. 7. Laser subsystem 130 includes a laser source 131 producing a visible aiming beam 132, and at least two therapeutic laser light sources 133a, 133b . . . 133n and emitting laser beams 134a, 134b . . . 34n, wherein one or more of the therapeutic laser light sources may be designed to operate at a lower power for procedures conducted on soft tissue, such as skin or gum tissue, and one or more of the other therapeutic laser light sources may be designed to operate at a higher power for procedures on hard tissue, such as tooth enamel, tooth dentin, or bone. In one such exemplary embodiment, laser light source 133a is a Neodymium/YAG or semiconductor diode laser having a power range adjustable between about 0 to about 15 Watts, such as from about 0.1 to about 15 Watts, and used for soft-tissue applications and laser light source 133b is an Erbium/YAG diode-pumped solid-state laser or a flashlamp-pumped solid-state laser used for hard-tissue applications. Laser beams 134 are collected in optical coupler 135 whereby any of laser beams 132 and 134 emit from a single location and exit laser subsystem 130 as beam 136.

In one exemplary embodiment, power supply 120 includes insulated-gate bipolar transistors, which may allow an operator or technician to set a variable pulse width for therapeutic laser light source 133a and/or therapeutic laser light source 133b, in order to modify the power yield as a function of time. For example, power supply 120 can be configured to provide high-power peaks of shorter duration to improve performance during hard-tissue ablation procedures. As a further example, power supply 120 can be configured with high repetition rates and medium duration pulses to cause cavitation within root canals to remove softer tissue and sterilize the interior of the canal. As another example, power supply 120 can be configured to provide longer duration power of lower peaks to improve comfort, consistency and/or quality of soft-tissue cutting and cauterizing procedures. In the illustrative embodiment, laser-emitting device 100 also includes foot pedal 195 that is operatively coupled to controller 140 using a wireless communication link. While foot pedal 195 uses a wireless link in the illustrative embodiment, it may also be operatively coupled to controller 140 using a wired connection.

By utilizing multiple therapeutic laser light sources, such as 133a, 133b . . . 133n, a wide variety of dental procedures may be performed on both soft-tissue and hard tissue. The list of soft-tissue procedures includes, but is not limited to, gingival troughing for crown impressions, gingivectomy and gingivoplasty, gingival incision and excision, soft-tissue crown lengthening, hemostatis and coagulation, excisional and incisional biopsies, exposure of unerupted teeth, fibroma removal, frenectomy and frenotomy, implant recovery, incision and drainage of abcess, leukoplakia, pulpotomy as an adjunct to root canal therapy, operculectomy, oral papilectomies, reduction of gingival hypertrophy, treatment of canker sores, herpetic and aphthous ulcers of the oral mucosa, and vestibuloplasty. Additional periodontal procedures include sulcular debridement, including removal of diseased, infected, inflamed and necrosed soft-tisuse in the periodontal pocket to improve clinical indices including gingival index, gingival bleeding index, probe depth, attachment loss and tooth mobility; laser soft-tissue curettage, laser removal of diseased, infected, inflamed and nectrotic soft-tissue within the periodontal pocket; removal of highly-inflamed edematous tissue affected by bacterial penetration of the pocket lining and junctional epithelium. The list of hard-tissue procedures includes, but is not limited to, laser drilling, bone ablation, tooth enamel and/or dentin ablation, and the desensitization of nerves within the tooth pulp by firing low power laser pulses through the relatively translucent tooth enamel and dentin. In addition, the use of laser light sources 133a and 133b allows laser-assisted whitening/bleaching of teeth and bio-stimulation of both hard-tissue and soft-tissue, as desired.

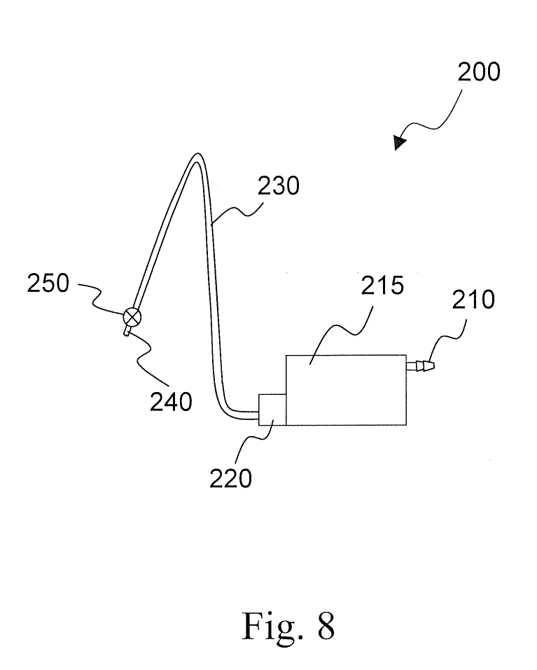

In one exemplary embodiment, as depicted in FIG. 8, airless misting unit 200 described above includes a water source 210, a reservoir 215, a high-pressure pump 220, a supply line 230, and an atomizing nozzle 240. Optionally, a check valve 250 may also be included to restrict the flow of water from nozzle 240 when airless misting unit 200 is not in operation. Atomizing nozzle 240 is designed to cause a fine mist of water to be ejected and mixed with the air present outside the nozzle when high-pressure pump 220 is activated by controller 140. By pressurizing the water in supply line 230 to from about 2 Bars to about 10 Bars of pressure (from about 30 psi to about 150 psi), such as from about 4 Bars to about 10 Bars or from about 4 Bars to about 8 Bars, air need not be introduced into supply line 230 to create the mist. In one exemplary embodiment, atomizing nozzle 240 includes orifices of between about 200 microns and about 500 microns and is manufactured by a laser drilling process which allows the airless mist generated by the unit to be optimized to provide efficient misting of the treatment area.

Conventionally, water spray or water mist for both the high speed air rotor handpieces and lasers was generated by combining liquid water and pressurized air. The liquid water and pressurized air were typically mixed in close proximity to a misting or spray orifice and fine particles of water were generated by the rapid expansion of the pressurized air as it escaped from the orifice. While effective for creating a water mist, the conventional technology necessitates two pressurized conduits, at least two meters in length, connected to the dental handpiece, and considerable expense and complexity associated with regulating the pressure to the liquid water and pressurized air. Furthermore, the requisite pressures were generated by pumps internal to the dental device or by connection to the pressurized air supply within a dental office.

In addition to the expense of regulating the air and water pressures within the dental unit or laser, operator error among dental office personnel could cause the air connection to the dental unit to be connected to a water supply in the dental operatory, with very damaging results.

Furthermore, dental offices were known to frequently have contaminated compressed air supplies due to water condensation during the compression process. The condensed water may be held in the compressed air tanks of a dental office for weeks or months and could become a breeding ground for bacteria, mold and other forms of contamination. Spraying contaminated water and air into open operative sites is a known source of infection and disease in the dental profession.

The airless misting system disclosed herein eliminates much of the complexity, expense, contamination risk and infection risk by producing a fine water mist or spray without the addition of compressed air. The use of a single, small high pressure water pump and a removable and cleanable water container allows the airless misting and improves the ease of operation of the laser system and also improve its safety.

As broadly disclosed herein, the airless mist is referred to water without any air added to it by way of addition of compressed air to the water. However, one of ordinary skill in the art will understand that any suitable liquid, without the addition of a compressed gas, may be used. One example of such a suitable liquid may be a medicament liquid. Any suitably liquid may be used so long as it is capable of cooling the treatment area and focusing the laser beam emitted by the disclosed device and also does not include any compressed or pressurized gas, such as air.

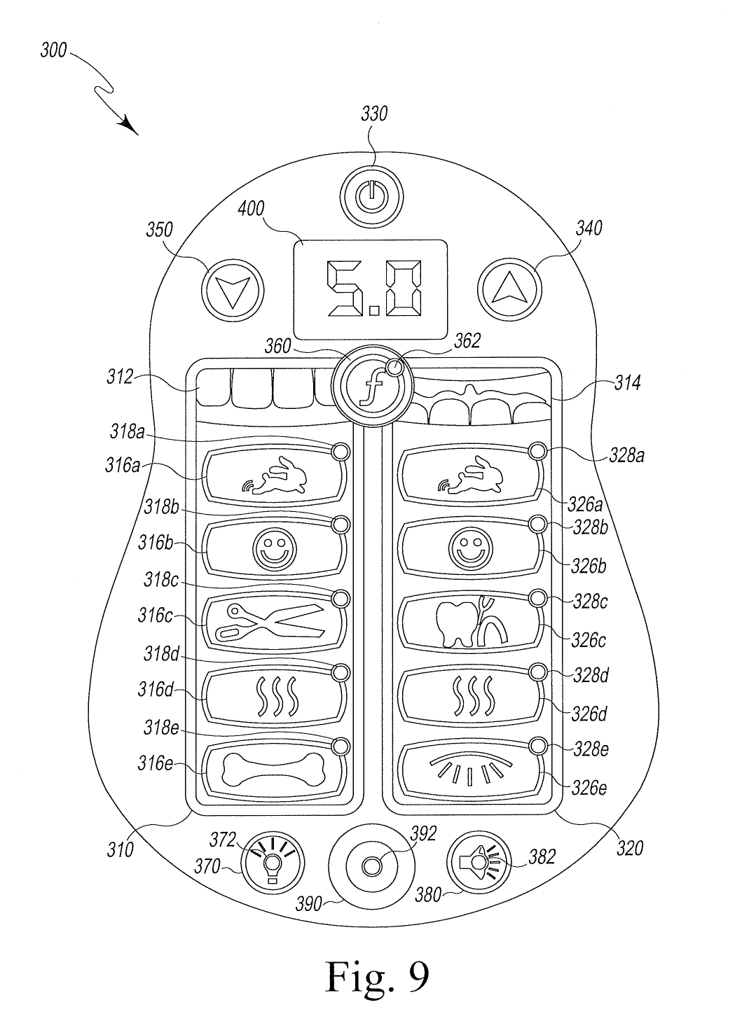

Referring now to FIG. 9, an exemplary embodiment of user interface 300 allows an operator of laser-emitting device 100 to quickly and easily select appropriate device settings. For example, an operator could select from an array of pre-programmed combinations of laser energy and pulse frequency by pressing a button or icon on user interface 300 that is associated with either the soft-tissue laser light source or the hard-tissue laser light source. In one such exemplary embodiment, each user-selectable button or icon causes the controller to set the laser energy and pulse frequency to a pre-determined setting stored in controller memory 150.

In the illustrative embodiment of FIG. 9, user interface 300 includes a bank 310 of user-selected buttons or icons 316 associated with pre-set parameters for the hard-tissue laser and a bank 320 of user-selected buttons or icons 326 associated with pre-set parameters for the soft-tissue laser. In the illustrative embodiment depicted in FIG. 9, bank 310 includes an icon 312 that indicates that it relates to the hard tissue laser operations, and bank 320 includes an icon 314 that it relates to the soft tissue laser operations. As further depicted in the illustrative embodiment of FIG. 9, bank 310 of user interface 300 includes five user-selectable buttons or icons 316a-e and bank 320 includes five user-selectable buttons or icons 326a-e.

In this illustrative embodiment, user interface 300 also provides additional buttons or icons and each button or icon may have its own corresponding indicator, such as an LED or similar device. Referring to FIG. 9, the following additional buttons/icons and indicators are depicted: on/off button or icon 330, up arrow button or icon 340, down arrow button or icon 350, “function” button or icon 360 with “function” indicator 362, light button or icon 370 with light indicator 372, sound button or icon 380 with sound indicator 382, and standby button or icon 390 with standby indicator 392. On/off button or icon 330 powers on or powers off laser-emitting device 100. Light button or icon 370 and sound button or icon 380 may be used to toggle one or more sound and visual indicators, respectively. Standby button or icon 390 places laser-emitting device 100 into or out of standby mode. Up arrow button or icon 340 and down arrow button or icon 350 allow a user to manually adjust the power settings from the pre-set parameters associated therewith. Furthermore, while bank 310 and bank 320 are each shown to include five buttons or icons in the illustrative embodiment, the number of buttons or icons associated with each bank is not limited thereto, but may encompass fewer or more buttons or icons, as necessary. In yet further embodiments (not pictured), the user interface may include an optional bank of buttons or icons directed to the control of endodontic procedures, such as preparing a tooth for and conducting a root canal. As one of ordinary skill in the art will understand, such additional buttons or icons for endodontic procedures may be placed on the user interface by any suitable method.

In the illustrative embodiment, bank 310 of the hard-tissue controls includes user-selectable button or icon 316a depicting a rabbit indicative of a “speed” setting; button or icon 316b depicting a “smiley face” indicative of a “comfort” setting; button or icon 316c depicting scissors indicative of a hard-tissue cutting or ablation setting; button or icon 316d depicting a set of wavy lines indicative of a “desensitization,” “decontamination,” or curettage setting; and button or icon 316e depicting a bone indicative of an osseous setting for ablating bone. In one such exemplary embodiment, the pre-set parameters associated with each button or icon of bank 310 indicates to controller 140 that the airless misting unit 200 should operate during operation of the hard-tissue laser. In the illustrative embodiment, indicators 318a-318e each corresponds to a user-selectable button or icon 316 to indicate the currently selected setting. In the illustrative embodiment show, indicators 318a-318e are depicted as light-emitting diodes that illuminate when each corresponding button or icon 316a-316e, respectively, is selected. For example, when button or icon 316a is selected by the user, indicator 318a changes to indicate the selection of that selection. While indicators 318 are depicted in FIG. 9 as light-emitting diodes (LEDs), they could also be elements of a Liquid Crystal Display (LCD), Organic Light Emitting Diodes (OLEDs) or other type of indicator capable of indicating information about the status of laser-emitting device 100. In another exemplary embodiment, indicators 318a-e are configurable icons on a touch-screen.

Similarly, in the illustrative embodiment, bank 320 includes five user-selected buttons or icons associated with pre-set parameters for the soft-tissue laser. As in the previous example, button or icon 326a depicting a rabbit indicates a “speed” setting for the soft-tissue laser; button or icon 326b depicting a smiling face indicates a “comfort” setting; button or icon 326c depicting a probe entering between a tooth and gum indicates a soft-tissue cutting or curettage setting; button or icon 326d depicting a set of wavy lines indicates a “desensitization” or “decontamination” or “curettage” setting; and button or icon 326e depicting lines emitting from a surface indicates a “tooth bleaching” or “bio-stimulation” setting. In one such exemplary embodiment, the pre-set parameters indicate to controller 140 that the airless misting unit 200 should not operate during operation of the soft-tissue laser. Furthermore, button or icon 326d could indicate to controller 140 that one set of laser parameters including pulse frequency and laser energy should be set, or button or icon 326d could be programmed to cycle through three or more different settings having different pulse frequencies and laser energy, but providing settings that are effective in one or more of the desensitization, decontamination or curettage procedures.

In the illustrative embodiment, indicators 328 each correspond to a user-selectable button or icon 326 to indicate the currently selected setting. In the illustrative embodiment shown, indicators 328 are depicted as light-emitting diodes that illuminate when each corresponding button or icon 326, respectively, is selected. For example, when button or icon 326a is selected by the user, indicator 328a changes to indicate the selection of the related pre-set laser parameters. While indicators 328 are depicted in FIG. 9 as light-emitting diodes (LEDs), they could also be elements of a Liquid Crystal Display (LCD), Organic Light Emitting Diodes (OLEDs) or other type of indicator capable of indicating information about the status of laser-emitting device 100. In another exemplary embodiment, indicators 328 are configurable icons on a touch-screen.

Visual display 400 indicates desired information about the status of at least one of laser light sources 133a. For example, in one such exemplary embodiment, visual display 400 indicates the operating power of therapeutic laser 133a corresponding to a selected setting when a button or icon from bank 310 has been selected, and visual display 400 indicates the operating power of therapeutic laser 133b corresponding to a selected setting when a button or icon from bank 320 has been selected. Other parameters may be shown on visual display 400, including pulse width, pulse frequency, or another laser parameter of interest to the operator. While visual display 400 is depicted in FIG. 9 as a multi-segment light-emitting diode (LED) display, it is not limited thereto. Visual display 400 could also be a Liquid Crystal Display (LCD), Organic Light Emitting Diode (OLED) or other type of display capable of indicating information about the status of at least one of laser light sources 133a. In another exemplary embodiment, visual display 400 is comprised of configurable icons on a touch-screen.

In another exemplary embodiment, a secondary visual display 410, as depicted in FIG. 1B, provides a visual indicator of a general status of the laser subsystem 130. The illustrative embodiment includes three cold-cathode tubes, wherein controller 140 causes a red cold-cathode tube 420 to illuminate to indicate that the laser-emitting device 100 is in soft-tissue mode, controller 140 causes a green cold-cathode tube 430 to illuminate to indicate that laser-emitting device 100 is in hard-tissue mode, and controller 140 causes a yellow cold-cathode tube 440 to illuminate to indicate that laser-emitting device 100 is in standby mode. Secondary visual display 410 provides a quick visual indication of the status of laser-emitting device 100 when an operator may by further away from the system or may not be able to see the other visual indicators. While red-, green- and yellow-colored cold-cathode tubes are used as secondary visual display 410 in this exemplary embodiment, other types and colors of light sources may be used, such as LEDs and OLEDs, or any other light-emitting devices of any color. In yet another exemplary embodiment, secondary visual display 410 is comprised of configurable icons or graphics on a touch-screen.

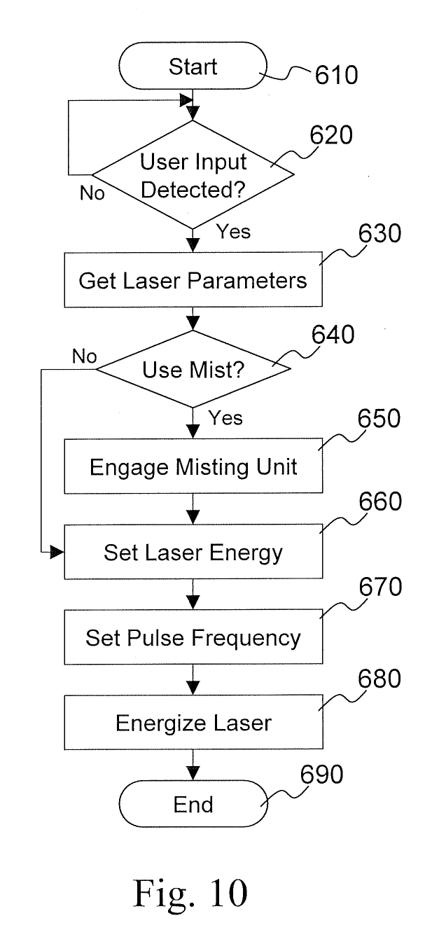

As described in the exemplary embodiment above, each button or icon in bank 310 and bank 320 may be configured to correspond to one or more pulse frequency/laser energy pre-set parameters. Moreover, in one exemplary embodiment, in addition to adjusting the laser parameters to the pre-set parameters in FIG. 10, controller 140 is configured to also engage airless misting unit 200 when one of the hard-tissue laser settings of bank 310 is selected, and controller 140 is configured to disengage airless misting unit 200 when one of the soft-tissue laser settings of bank 320 is selected.

Furthermore, while reference is made to an operator utilizing bank 310 and bank 320 of buttons or icons to select pre-set parameters for the laser-emitting device 100, an operator may also make selections on smart device 170 through buttons or icons. In one exemplary embodiment, the screen of smart device 170 mimics user interface 300 to provide a second method of selecting an operating mode of laser-emitting device 100.

In addition, smart device 170 may provide alternate methods of selecting an operating mode of laser-emitting device 100. In one such exemplary embodiment, smart device 170 is configured to use speech recognition to detect a verbal command of an operator and communicate with controller 140 to select the applicable pre-set parameters. For example, smart device 170 may listen for the operator to speak verbal commands, such as “soft tissue speed” or “hard tissue comfort,” in response to which smart device 170 would communicate the selection to controller 140 which would make the corresponding selection of pulse frequency and laser energy and would update user interface 300, visual display 400, and secondary visual display 410. In addition, smart device 170 could also be configured to respond with synthesized speech output to provide an auditory confirmation of the selected operating mode of laser-emitting device 100, regardless of whether the selection was made by voice or through the user interface.

Additional functionality is provided by smart device 170. In one exemplary embodiment, smart device 170 not only communicates with controller 140, but is also designed to communicate with other systems apart from laser-emitting device 100. A variety of applications exist for such two-way communication. For example, a diagnostic program designed to run on smart device 170 could diagnose laser system 100 based upon operating parameters and/or usage data and transmit that information back to the manufacturer of laser-emitting device 100, or to a third-party service company, to assist in troubleshooting and repair of a malfunctioning unit.

In another exemplary embodiment, smart device 170 would receive software and/or firmware updates from the manufacturer and upgrade laser-emitting device 100. In yet another exemplary embodiment, smart device 170 could calibrate one or more of the lasers 132 and/or 133 utilizing two-way communication between the manufacturer and laser-emitting device 100. For example, the manufacturer could initiate an upgrade to the laser system 100 through communication with smart device 170 to program power supply 120 to operate at a different pulse width profile based either on new data available to the manufacturer or at the request of the user of laser-emitting device 100.

In yet another exemplary embodiment, an operator of smart device 170 could initiate a chat, email communication, or online help resource to receive support. In yet another exemplary embodiment, an operator of smart device 170 could order accessories, consumables, new products or upgrade to a newer version of laser-emitting device 100.

Although, in the illustrative embodiment, smart device 170 is described and depicted as an Apple iPad™, it is not limited thereto. For example, smart device 170 could take the form of any brand of cellular telephone including, but not limited to, an Apple brand iPhone™ cellular telephone, Droid™ cellular telephone or Blackberry™ cellular telephone. Smart device 170 could also be a tablet computer (or tablet-like computer) of any screen size and capable of being operatively coupled to laser-emitting device 100 via a wired or wireless connection.

Further, while in one exemplary embodiment smart device 170 is described as having wireless communication capability compatible with an IEEE 802.11 standard (“WiFi” or “WiFi Direct”), any wireless communication standard is considered within the scope of the present invention. Other examples of wireless communication capability include, but are not limited to, CDMA, W-CDMA, GSM, 3G or 4G, or WiMAX communication protocols, or any other appropriate wireless communication protocol.

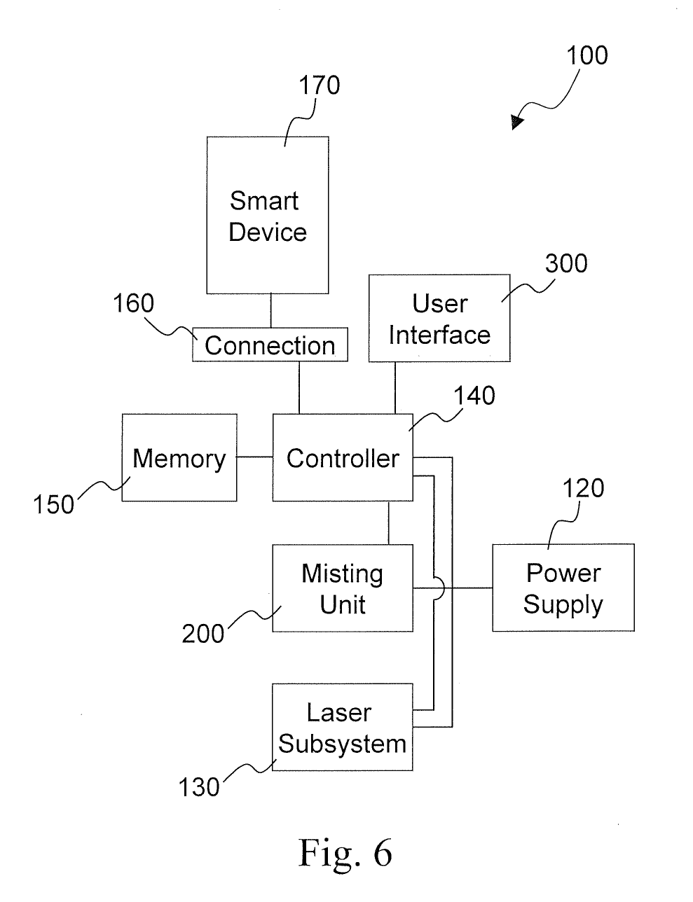

Similarly, although the exemplary embodiment depicted in FIGS. 1-6 illustrates smart device 170 as physically connected to connection 160 in a “docked configuration,” the invention is not necessarily limited to that connection type and could also be connected via a cable (not shown) or a wireless connection, such as IEEE 802.11 WiFi, WiFi Direct, Bluetooth, WiMAX, or any other appropriate wireless communication protocol.

Referring now to FIG. 10, a method of operating a dental laser-emitting device is described. After the method begins in step 610, laser-emitting device detects whether a user has interacted with the user interface to select an operating mode in step 620. Upon detection of user input, in step 630 the controller retrieves the laser parameters associated with the selected operating mode. One of the parameters includes whether airless misting should be administered, which the method determines in step 640. If the laser parameters for a certain operating mode require airless misting, the airless misting unit is engaged in step 650. Either when the airless misting is determined to not be required in step 640 or after the airless misting unit is engaged in step 650, the controller sets the laser energy in step 660 to match the selected parameters retrieved in step 630. Similarly, in step 670, the controller sets the laser pulse frequency to match the parameters retrieved in step 630. Upon setting the laser energy and pulse frequency, the controller energizes the laser in step 680 and the routine ends in step 690.

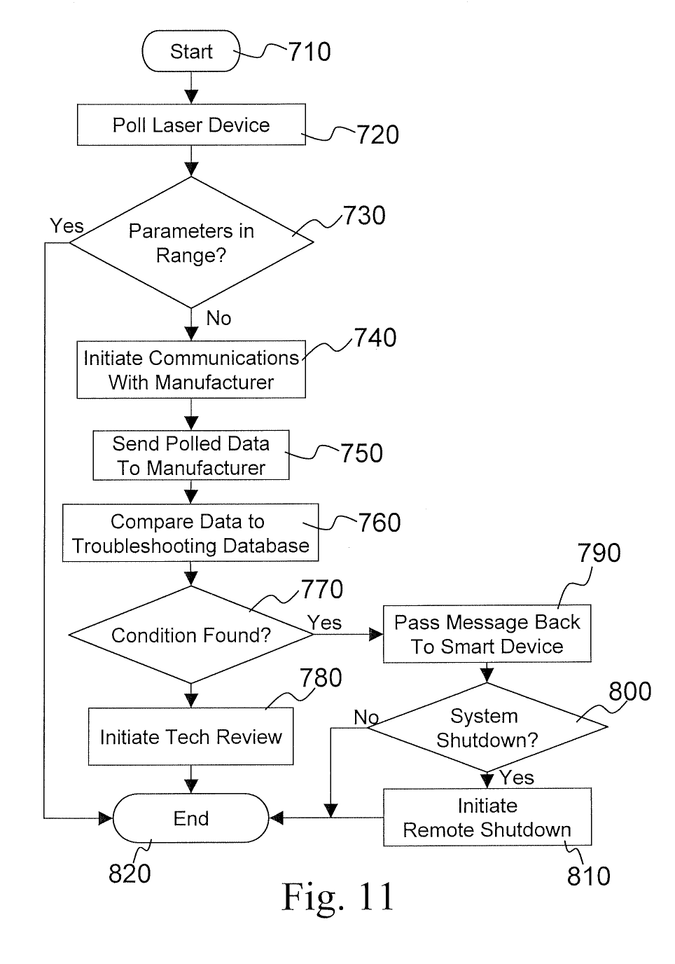

Referring now to FIG. 11, a method of performing remote diagnostics and/or telemetry of a dental laser-emitting device is described. After the routine begins in step 710, the controller or smart device polls the laser device in step 720 to record operating parameters, such as pulse energy, pulse frequency, pulse width, number of flash-lamp pulses fired, number of laser pulses fired, hours of laser operation in standby mode, hours of laser operation in ready mode, hours of laser operations in operational mode (laser actually firing), coolant temperature, laser head temperature, air temperature within the device, or any other measurable parameter of interest. If the parameters are in a specified range, as determined in step 730, the diagnostic and/or telemetry routine ends in step 820. However, should the parameters retrieved in step 720 be outside of the specified range, the smart device initiates communications with the device manufacturer or a third-party service company in step 740. In one exemplary embodiment, the communications between the smart device is initiated through a wireless connection to the internet, such as through an IEEE 802.11 standards-based wireless protocol. Another method of connection may also be used, including Bluetooth, CDMA, GMA, 3G, 4G or any suitable method for initiating a connection to the manufacturer or third-party service company.

After the connection is established, the smart device sends the data polled in step 720 to the manufacturer in step 750. A web-enabled server associated with the manufacturer reads the data provided through the communication channel and compares it to that stored in a troubleshooting database in step 760. If the data provided does not match a condition found in the troubleshooting database, in step 770 the web-enabled server initiates a technician review. This can be done in a variety of ways, including by sending an email message to a technician, creating an entry in a service database, sending a text message to a computer or cellular device, or any other known method of sending a message between a web-enabled server and a user, after which the diagnostic and/or telemetry routine ends in step 820. While a web-enabled server is described in the illustrative embodiment, a similar device capable of communication and assessment of the polled data may also be used.

However, should the data provided in step to the web-enabled server in step 750 match a condition found in the troubleshooting database, the web-enabled server in step 790 transmits a message back to the smart device. Such message may be sent through the same communications method as the original message sent from the smart device to the web-enabled server. In addition, other communications could be sent in step 790. In one exemplary embodiment, an email message is transmitted to a distribution list associated with the web-enabled server or similar device. In another exemplary embodiment, an automated phone call is placed to a telephone number or numbers associated with the web-enabled server. In yet another exemplary embodiment, a technician receives a message to contact the operator registered to the dental laser-emitting device to discuss the detected condition.

In another exemplary embodiment, the data polled in step 720 is used to facilitate routine, preventative and/or predictive maintenance. For example, the communication described in step 790 may include instructions to replace the flash-lamp after a certain number of pulses is reached, to alert the user to change a filter after a certain number of hours of standby, ready, or operational time has passed. While these examples are provided for illustrative purposes, any routine, preventative, or predictive maintenance may be initiated based upon the data polled in step 720, and it is not limited to the examples provided.

In certain instances, it may be desirable to shut down the dental laser-emitting device when parameters vary outside of a normal range. In the illustrative embodiment, the diagnostic method determines in step 800 that the dental laser-emitting device should be shut clown for safety reasons. Once that determination is made, a remote shutdown is initiated in step 810 by sending a command from the web-enabled server to the smart device. Once the command is received by the smart device, the diagnostic program ends in step 820 and the dental laser-emitting device is shut down. In one exemplary embodiment, other activities are triggered by the remote system shutdown, such as the initiation of a service call for the malfunctioning dental laser-emitting device. Said remote diagnostics within the smart device may provide redundancy and back-up to the safeguards and “watchdog” routines within the laser operating software. Should an error condition be detected, the smart device is capable of overriding the control of the laser and shutting the system down—thus providing greater safety for the operator and the patient.

Although the invention has been described in detail with reference to certain illustrated exemplary embodiments, variations and modifications exist within the scope and spirit of the invention.

We claim:

1. A laser-emitting device comprising:
a housing,
a power supply,
a laser subsystem having two or more laser light sources and an aiming beam,
a controller configured to modulate one or more of the laser light sources;
a memory operatively coupled to the controller to store device settings;
a connection used to operatively couple a smart device to the controller;
a handpiece for applying laser light to the area of treatment; and
an airless misting system which does not require connection to an air supply,
wherein at least one laser light source is capable of soft tissue applications and at least one other laser light source is capable of hard tissue applications.

2. The laser-emitting device according to claim 1, wherein the airless misting unit is capable of applying a fine water mist to the area of treatment without addition of compressed air.

3. The laser-emitting device according to claim 1, wherein the airless misting unit generates pressures internal to the laser-emitting device of from about 2 Bar to about 10 Bar in liquid water.

4. The laser-emitting device according to claim 4, wherein the pressure is generated by a DC powered electrical pump.

5. The laser-emitting device according to claim 1, further comprising a single pressurized conduit connecting the handpiece to a high-pressure pump.

6. The laser-emitting device according to claim 1, further comprising a pressure regulating portion that regulates the pressure within the liquid water, wherein the pressure regulating portion is controlled by a digital or analog control circuit within the light-emitting device.

7. The laser-emitting device according to claim 1, further comprising a nozzle or orifice incorporated into a single use handpiece sleeve or handpiece cover such that the need for handpiece sterilization between uses or between patients is eliminated.

8. The laser-emitting device according to claim 1, further comprising an articulated arm operatively coupling the laser light source to the handpiece.

9. The laser-emitting device according to claim 1, wherein the at least one laser light source capable of soft tissue applications is a semiconductor diode laser or a neodymium/yttrium-aluminum-garnet diode laser.

10. The laser-emitting device according to claim 9, wherein the semiconductor diode laser has power range adjustable from about 0 Watts to about 15 Watts.

11. The laser-emitting device according to claim 1, wherein the other light source capable of hard tissue applications is an Erbium/yttrium-aluminum-garnet diode-pumped solid state laser or a flashlamp-pumped solid-state laser.

12. The laser-emitting device according to claim 1, wherein the laser subsystem further includes an alternative light source producing a visible aiming beam.

13. The laser-emitting device according to claim 1, wherein soft tissue applications are gingival troughing, gingivectomy and gingivoplasty, gingival incision and excision, soft-tissue crown lengthening, hemostatis and coagulation, excisional and incisional biopsies, exposure of unerupted teeth, fibroma removal, frenectomy and frenotomy, implant recovery, incision and drainage of abcess, leukoplakia, pulpotomy as an adjunct to root canal therapy, operculectomy, oral papilectomies, reduction of gingival hypertrophy, treatment of canker sores, herpetic and aphthous ulcers of the oral mucosa, and vestibuloplasty, sulcular debridement, laser soft-tissue curettage, laser removal of diseased, infected, inflamed and/or necrotic soft-tissue within the periodontal pocket; or removal of highly-inflamed edematous tissue affected by bacterial penetration of the pocket lining and junctional epithelium.

14. The laser-emitting device according to claim 1, wherein hard tissue applications are laser drilling, bone ablation, tooth enamel and/or dentin ablation, or the desensitization of nerves within the tooth pulp by firing low power laser pulses through the relatively translucent tooth enamel and dentin.

15. The laser-emitting device according to claim 1, wherein the at least one other laser light source capable of hard tissue applications is also capable of root canal procedures.

16. The laser-emitting device according to claim 1, wherein a foot pedal is operatively coupled to the controller via a wireless or wired communication link.

17. A method of operating a dental laser-emitting device in order to treat a patient, the method comprising:
a user selects an operating mode in a user interface of the laser-emitting device, where the operating mode is a soft tissue application or a hard tissue application,
a controller retrieves laser parameters associated with the selected operating mode,
the controller determines whether optional airless misting is required based upon the laser parameters associated with the selected operating mode,
following the determination of whether airless misting is necessary, the controller sets a laser energy and laser pulse frequency to match the selected parameters,
upon setting the laser energy and laser pulse frequency, the controller enables the laser-emitting device,
the user directs the enabled laser-emitting device to a treatment site of the patient to be treated, and
the user activates or energizes the laser-emitting device to begin treatment at the treatment site.

18. The method according to claim 17, wherein the laser-emitting device includes at last one laser light source capable of the soft tissue applications.

19. The method according to claim 18, wherein the at least one laser light source capable of soft tissue applications is a semiconductor diode laser or a neodymium/yttrium-aluminum-garnet diode laser.

20. The method according to claim 19, wherein the semiconductor diode laser has power range adjustable from about 0 Watts to about 15 Watts.

21. The method according to claim 17, wherein the laser-emitting light source includes at least one other laser light source capable of the hard tissue applications.

22. The method according to claim 21, wherein the other light source capable of hard tissue applications is an Erbium/yttrium-aluminum-garnet diode-pumped solid state laser or a flashlamp-pumped solid-state laser.

23. An airless misting system for use with a dental device, the airless misting system comprising:
a dental unit,
a dental handpiece,
a pressure generator in the dental unit that generates a pressure of from about 2 Bar to about 10 Bar in a liquid,
a pressure regulator in the dental unit that regulates the pressure within the liquid in the dental device,
a solenoid or electrical valve in the dental unit that starts and stops the flow of pressurized liquid from the dental unit to the dental handpiece,
a pressurized flexible conduit that connects the dental unit to the dental handpiece,
a nozzle or orifice in the handpiece that generates a fine liquid mist form the pressurized liquid in the direction of an area of treatment.

24. The airless misting system according to claim 23, wherein the pressure regulator is controlled by a digital or analog control circuit within the dental unit.

25. The airless misting system according to claim 23, wherein the nozzle or orifice are incorporated into a single use handpiece sleeve or handpiece cover.

26. The airless misting system according to claim 23, wherein the dental device is a laser-emitting device capable of treating hard tissue or soft tissue at the area of treatment.
